(19) United States
(12) Patent Application Publication
(10) Pub. No.: US 2018/0147332 A1
(43) Pub. Date: May 31, 2018

(54) SYSTEM FOR VENTRICULAR CIRCULATORY SUPPORT

(71) Applicants: Stephan Ensminger, Marloffstein (DE); Tim Kaufmann, Aachen (DE)

(72) Inventors: Stephan Ensminger, Marloffstein (DE); Tim Kaufmann, Aachen (DE)

(21) Appl. No.: 15/560,550

(22) PCT Filed: Mar. 23, 2016
(86) PCT No.: PCT/EP2016/056327

§ 371 Date: Jan. 16, 2018

(30) Foreign Application Priority Data
Mar. 23, 2015 (EP) 15160380.0

(51) Int. Cl. A61M 1/10; A61M 1/12

(52) U.S. Cl. CPC A61M 1/1055; A61M 1/122; A61M 1/125

(57) ABSTRACT

The invention relates to a system for ventricular circulatory support, comprising a device (2) for applying a magnetic field to a magnetised body fluid (1), particularly blood, in a region of a vessel (3), particularly a blood vessel. The system also comprises a control device designed to actuate said device (2) for applying a magnetic field such that, in the region of the vessel (3), a magnetically-induced force acts on the magnetised body fluid (1) in the longitudinal direction of said vessel (3).

### CROSS-REFERENCE TO RELATED APPLICATIONS

This application is a national phase of PCT Application Number PCT/EP2016/056327 filed Mar. 23, 2016, which claims priority to European Patent Application 15160380.0 filed Mar. 23, 2015, the entire contents both of which are hereby incorporated by reference herein.

### TECHNICAL FIELD

The invention relates to a system for ventricular circulatory support.

Known systems for ventricular circulatory support, in particular artificial hearts, are designed as mechanical pumps. Here various systems are used, for example, centrifugal pumps, continuously operating radial or axial pumps, pulsatile positive displacement pumps etc., wherein rigid mechanical connections or pneumatic connections are known for driving said pumps. These known systems are used to support the heart generally either as a left ventricular assist device (LVAD) or as a right ventricular assist device (RVAD).

In extreme cases, both ventricles have to be supported (biventricular assist device (BiVAD)). Upon failure of the natural heart, it may be necessary to provide an artificial heart as a complete replacement of the natural heart (total artificial heart: TAH). Here, the natural heart of the patient is fully explanted and replaced by a corresponding mechanical pump in one operation. Alternatively, the artificial heart can be extracorporeally provided.

Known systems have the disadvantages that the mechanical components of the artificial heart are subjected to wear and friction both in a supportive artificial heart as well as in an artificial heart which is provided for the complete replacement of the natural heart. As a result, the service life of such a system is limited and such an artificial heart has to be replaced after a relatively short time. The reliability of such an artificial heart is also compromised, which can be life threatening for a patient in the case of a failure of the artificial heart.

In addition, a strict anticoagulation (blood thinning) of patients is necessary for all currently available artificial hearts as a result of blood contact with the artificial surface of the system. This is done to prevent blood clots from forming but also is responsible for a substantial proportion of complications, such as, e.g., stroke or brain hemorrhage during treatment with an artificial heart system.

### SUMMARY

The aim of the invention is therefore to specify a system for ventricular circulatory support which is more reliable and durable.

The inventive aim is met by the subject matter of patent claim 1. Advantageous modifications to the invention are specified in the dependent patent claims.

Accordingly, the invention relates to a system for ventricular circulatory support, comprising a device for applying a magnetic field to a magnetized body fluid, particularly blood in a region of a vessel, particularly a blood vessel. The system also comprises a control device designed to actuate said device for applying a magnetic field such that, in the region of the vessel, a magnetically-induced force acts on the magnetized body fluid in the longitudinal direction of said vessel.

The advantages of the invention are obvious. While wear is inevitable in mechanical systems as a result of the constantly required pump output of the ventricular circulatory support system and the operating reliability is at risk, the wear in comparison to known systems is reduced in the inventive system for ventricular circulatory support because no moving mechanical elements are necessary. For the same reason, the operating reliability can be increased because no mechanical and particularly mechanically movable parts of the system for ventricular circulatory support have to be moved.

In particular, the solution according to the invention is characterized in that the system for ventricular circulatory support can be arranged outside of the blood vessel and thus operates entirely without direct blood contact. Hence, anticoagulation measures (blood thinning) can be omitted.

Provision is made according to one aspect of the invention for the control device to be additionally designed to temporally vary the strength and/or direction of the magnetic field applied to the magnetized body fluid in such a way that the body fluid optionally moves continuously or in a pulse-like manner in the longitudinal direction of the vessel. In addition, a helical movement of body fluids is also possible.

A continuous flow behavior of the body fluid can ensure that the body of a patient is uniformly supplied with said body fluid. Particularly in the case in which the body fluid relates to blood, it can be ensured that all body parts of the patient are evenly supplied with blood. The continuous flow of blood in this case also leads advantageously to the vessels being relieved especially of recurring pressure fluctuations acting on the vessel walls.

In this context, it should be noted that not only blood can be moved through the ventricular circulatory support system. For example, the body fluid can also relate to lymph fluid.

Alternatively, the control device can also temporally vary the magnetic field applied to the magnetized body fluid such that the body fluid is moved in a pulse-like manner in the longitudinal direction of the vessel. In this case, the natural functionality of a natural heart is simulated. It can thus be ensured that all functions of the body of a patient can continue to be operated as usual because the system for ventricular circulatory support functionally replaces the natural heart of a patient.

According to a further aspect of the invention, provision is made for the system for the ventricular circulatory support to further comprise magnetizing means for magnetizing a body fluid, in particular blood.

This ensures that the body fluid can be optimally moved by means of the application of the magnetic field, i.e. that a force can be transferred to the body fluid in an optimized manner.

In this context, it should be noted that the body fluids of a patient, in particular blood, comprise corpuscular constituents, such as, e.g., erythrocytes. By applying a magnetic field, a magnetic force can then be particularly exerted if the corpuscular constituents were magnetized.

Provision is made according to a further aspect of the invention for the magnetizing means to be designed to inject magnetized or magnetizable particles into the body fluid.

This allows for the magnetization of the body fluid to be increased, in particular by means of the magnetization of constituents contained in the body fluid.

It is conceivable here that, for example, magnetized or magnetizable particles, for example iron-containing particles, are at least temporarily deposited on cells contained in the body fluid. This deposition can, for example, be carried out by means of a chemical process. It is also conceivable that such a deposition can be achieved by virtue of the fact that the surface of the magnetic or magnetizable particles is modified in such a way that these particles “dock” to the cells contained in the body fluid. It is also conceivable here that a bond forms analogous to the key-lock principle similar to the bond of antibodies to corresponding viruses.

On the other hand, it is conceivable that the magnetic or magnetizable particles are formed as nanoparticles and are, for example, enclosed within cells located in the body fluid. It is also, of course, conceivable that the magnetic or magnetizable particles are provided free in the blood plasma or respectively in the body fluid.

According to a further aspect of the invention, provision is made for the magnetizing means to be designed to treat the body fluid in such a way that a dipole moment is formed in the case of constituents of the body fluid.

The formation of a dipole moment makes it is possible to exert a magnetic force on precisely these constituents of the body fluid by applying a magnetic field.

According to a further aspect of the invention, provision is made for the magnetizing means to be designed to intracorporeally magnetize the body fluid, in particular by means of oral administration or by means of intravenous injection of magnetized or magnetizable particles.

It is conceivable in this case that magnetized or magnetizable particles are administered in tablet form or are injected by means of a syringe. Preferably the particles are supplied directly to the bloodstream of a patient by means of intravenous injection so that the blood of the patient is magnetized or magnetizable within a short time.

According to a further aspect of the invention, provision is made for the magnetizing means to be designed to extracorporeally magnetize the body fluid, in particular by injection or diffusion of magnetized or magnetizable particles.

It is conceivable here that the magnetization of the body fluid is carried out, for example, during a dialysis of the blood of a patient. For this purpose, a dialysis machine can be used or alternatively a dialysis machine can be modified so that this dialysis machine is set up to magnetize the body fluid of a patient. On the other hand, it is, of course, conceivable that a dialysis machine is not used for this purpose but another machine which is designed to inject magnetizable or magnetized particles into the blood of a patient or to magnetize the body fluid of a patient by means of diffusion of magnetized or magnetizable particles into the body fluid of the patient.

According to a further aspect of the invention, provision is made for the device for applying a magnetic field to have a magnetic coil assembly at least in certain regions in the longitudinal direction of the vessel, said magnetic coil assembly being segmented into a plurality of regions that are successive and can be actuated independently of one another.

The magnetic coil arrangement consists preferably of at least one electromagnet, which is designed to generate a magnetic field when activated, which is suitable to accelerate at least one particle comprising a dipole moment within the body fluid of a patient in the longitudinal direction of the vessel.

According to a further aspect of the invention, provision is made for the control device to be designed to successively actuate the individual regions of the magnetic coil assembly in the direction of flow of the body fluid in such a way that a magnetic field is applied in each case by a single region alone to the magnetized fluid.

This ensures that particles which are contained in the body fluid of the patient and which have a dipole moment can be accelerated in the direction of flow and thus the result is that the body fluid of the patient is moved in the direction of flow of the vessel. Here, the effect is utilized that the body fluid as a whole is set in motion by means of internal friction even if only individual constituents which are contained in the body fluid are accelerated. In other words, the constituents that have a dipole moment and are accelerated by means of the magnetic coil assembly “pull” the remaining constituents of the body fluid that are not magnetized along, so that in total a movement of the body fluid of a patient is realized in the direction of flow of the vessel.

According to a further aspect of the invention, provision is made for the individual regions of the magnetic coil assembly to be spatially separated from one another, and wherein, as seen in the direction of flow of the body fluid, the first region of the magnetic coil assembly can be actuated by the control device in order to apply a magnetic torque to magnetized or magnetizable particles disposed upstream of the first region, and wherein a further region of the magnetic coil assembly, which is disposed downstream with regard to the first region of the magnetic coil assembly, is actuated by the control device if the magnetized or magnetizable particles were moved by means of the magnetic field of the first region of the magnetic coil assembly along the vessel in the direction of the first region of the magnetic coil assembly.

In this way, the constituents of the body fluid which have a dipole moment are subjected multiple times to an acceleration and preferably to the same acceleration. It is conceivable that a linear acceleration is hereby exerted on the magnetic or magnetizable constituents of the body fluid. In other words, the regions of the magnetic coil assembly can be actuated such that the body fluid is linearly and uniformly accelerated; thus enabling a continuous force to be exerted on the magnetized or magnetizable constituents of the body fluid. Hence, a constant pump output is generated.

On the other hand, the regions of the magnetic coil assembly can be actuated such that a pulse-like or wave-like force is exerted on the magnetic or magnetizable constituents of the body fluid. As a result, the natural functionality of a heart can be simulated.

According to a further aspect of the invention, provision is made for the device for applying a magnetic field to have a large number of magnetic coil assemblies in successive vessel sections, wherein a magnetic field applied by the device is generated along the successive vessel sections in such a way that a magnetic force, which has a component pointing in the longitudinal direction of the vessel, is exerted on magnetized or magnetizable particles in the body fluid.

This allows for a single magnetic coil assembly to be dimensioned smaller and a comparable ventricular circulatory support to be achieved at the same time.

### BRIEF DESCRIPTION OF THE DRAWINGS

The invention is described below in greater detail with reference to the accompanying drawings using exemplary embodiments of the system according to the invention for ventricular circulatory support.

In the drawings:

FIG. 1: shows a schematic sectional view of the system according to the invention for ventricular circulatory support, of a vessel as well of a magnetic or magnetizable particle;

FIG. 2: shows a schematic sectional view of the system according to the invention for ventricular circulatory support, of a vessel as well as of a magnetic or magnetizable particle with exertion of a magnetic force by means of a second region of a magnetic coil assembly;

FIG. 3: shows a schematic sectional view of the system according to the invention for ventricular circulatory support, of a vessel as well as of a magnetic or magnetizable particle with exertion of a magnetic force by means of a third region of a magnetic coil assembly; and

FIG. 4 shows a schematic sectional view of the system according to the invention for ventricular circulatory support, of a vessel as well as of two magnetic or magnetizable particles.

### DETAILED DESCRIPTION

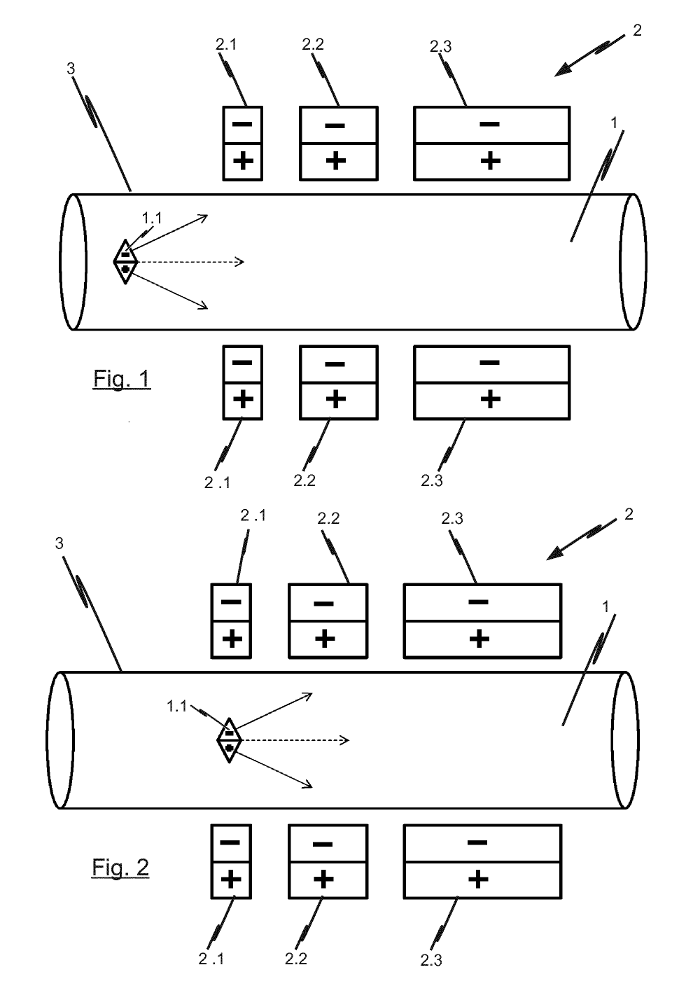

FIG. 1 shows a device 2 for applying a magnetic field to a magnetized body fluid 1. The device 2 is designed as a magnetic coil assembly having three exemplary regions 2.1, 2.2, 2.3, which surround a vessel 3.

Within the vessel 3, there is a constituent 1.1, such as a magnetic or magnetizable particle or a magnetized or magnetizable cell, of a body fluid 1 of a patient. Only one single such constituent is schematically shown in FIG. 1. Many constituents that are magnetized or magnetizable are normally contained within the body fluid 1 of a patient.

The magnetic coil assembly 2 is schematically composed of three regions 2.1, 2.2, 2.3. It is, of course, conceivable that the magnetic coil assembly 2 has only one region or alternatively more than three regions at its disposal. In FIG. 1, the case is schematically depicted where a constituent 1.1 that is magnetized or magnetizable and is provided in the body fluid 1 of a patient moves in the proximity of the magnetic coil assembly.

A first region 2.1 of the magnetic coil assembly 2 is actuated by a control device (not depicted) in such a way that the constituent 1.1 radiates a force in the longitudinal direction and in the direction of flow of the vessel 3. As depicted in FIG. 1, a magnetic force from the minus pole of the dipole of the constituent 1.1 onto the plus pole of the first region 2.1 of the magnetic coil assembly 2 and a force from the plus pole of the constituent 1.1 onto the minus pole of the first region 2.1 of the magnetic coil assembly 2 act in this case on the constituent 1.1, which is magnetized or magnetizable.

In this context, it should be noted that the terms “minus pole” and “plus pole” are to be understood as the magnetic north or respectively south pole.

From the magnetic forces (arrows having solid lines, starting at constituent 1.1 in FIG. 1) acting on the constituent 1.1, a resulting force (arrow having a dashed line, starting at constituent 1.1 in FIG. 1) is exerted on the constituent 1.1, which generates an acceleration of said constituent 1.1 in the direction of flow within the vessel 3.

The case is shown in FIG. 2, where the constituent 1.1 was accelerated in the direction of flow within the vessel 3 and now the magnetic coil assembly 2 is actuated in such a way that the second region 2.2 of the magnetic coil assembly 2 is actuated so that, as already described in reference to FIG. 1, now a force is exerted on the magnetic or magnetizable constituent 1.1 in the body fluid 1 in the longitudinal direction or respectively in the direction of flow of the vessel 3 correspondingly from the second region 2.2 of the magnetic coil assembly 2.

FIG. 3 shows the corresponding case in which the magnetic or magnetizable constituent 1.1 was accelerated within the body fluid 1 of the patient further in the direction of flow of the vessel and now the third region 2.3 of the magnetic coil assembly 2 is actuated by the control device, so that said third region 2.3 of the magnetic coil assembly 2 exerts a magnetic force on the magnetic or magnetizable constituent 1.1 in the direction of flow of the vessel 2.

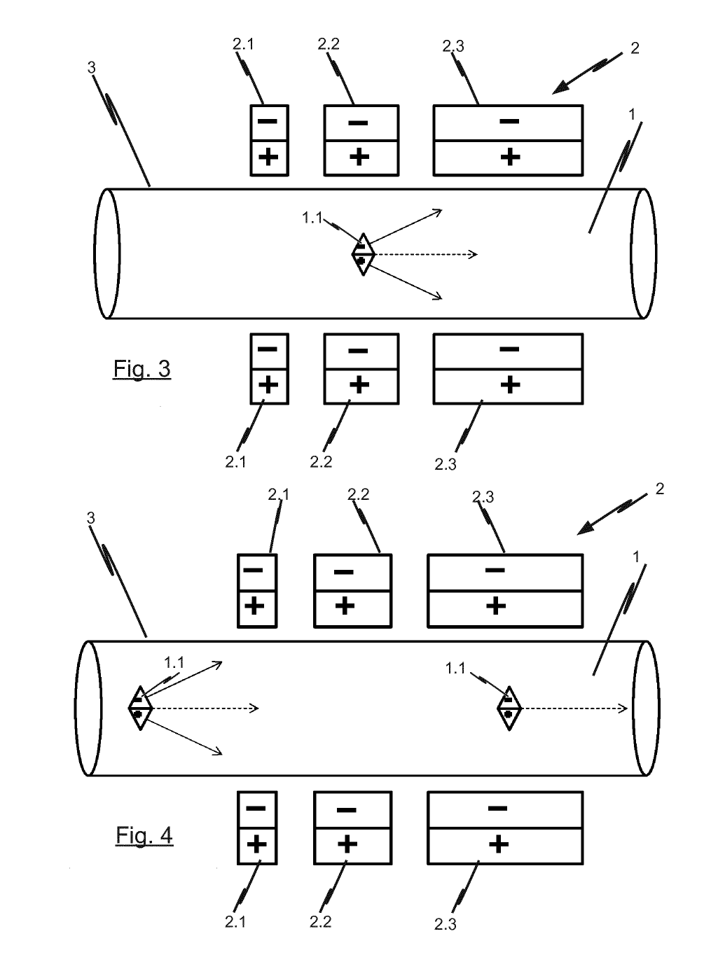

FIG. 4 shows in turn the case where a further magnetic or magnetizable particle 1.1 or respectively a further magnetic or magnetizable constituent 1.1 of the body fluid 1 of the patient approaches the magnetic coil assembly 2 and a corresponding control device actuates the regions 2.1, 2.2, 2.3 of the magnetic coil assembly 2 so that this constituent 1.1 is also accelerated in the direction of flow of the vessel 3.

In other words, the magnetic coil assembly 2 is functionally actuated in such a way that the constituents 1.1 of the body fluid 1 of the patient are preferably accelerated linearly in the direction of flow of the vessel 3.

On the other hand, the regions 2.1, 2.2, 2.3 of the magnetic coil assembly 2 can be actuated in such a way that a pulse-like acceleration of the constituents 1.1 within the body fluid 1 of the patient is generated in the direction of flow of the vessel 3.

The invention is not limited to the embodiments of the system for ventricular circulatory support depicted in the drawings but ensues from an overview of all the features disclosed herein.

### REFERENCE NUMBER LIST

1 magnetized body fluid

1.1 constituent/particle

2 device P/magnetic coil assembly

2.1, 2.2, 2.3 regions of the magnetic coil assembly

3 vessel

1. System for ventricular circulatory support, the system comprising:
a device (2) for applying a magnetic field to a magnetized body fluid (1), in a region of a vessel (3); and
a control device designed to actuate said device (2) for applying a magnetic field such that, in the region of the vessel (3), a magnetically-induced force acts on the magnetized body fluid (1) in the longitudinal direction of said vessel.

2. System according to claim 1, wherein the control device is further designed to temporally vary the strength and/or direction of the magnetic field applied to the magnetized body fluid (1) such that the body fluid (1) moves in a pulse-like manner in the longitudinal direction of the vessel (3).

3. System according to claim 1, which further comprises a magnetizing means for magnetizing the body fluid (1).

4. System according to claim 3, wherein the magnetizing means is designed to inject magnetized or magnetizable particles (1.1) into the body fluid (1).

5. System according to claim 3, wherein the magnetizing means is designed to treat the body fluid (1) in such a way that a dipole moment is formed in the case of constituents (1.1) of the body fluid (1).

6. System according to claim 3, wherein the magnetizing means is designed to extravascularly, intravascularly or intracorporeally magnetize the body fluid (1) by means of oral administration or by means of intravenous injection of magnetized or magnetizable particles (1.1).

7. System according to claim 3, wherein the magnetizing means is designed to extracorporeally magnetize the body fluid (1) by injection or diffusion of magnetized or magnetizable particles (1.1).

8. System according to claim 1, wherein the device (2) for applying a magnetic field has a magnetic coil assembly disposed at least in certain regions in the longitudinal direction of the vessel (3), said magnetic coil assembly being segmented into a plurality of successive regions (2.1, 2.2, 2.3) that can be actuated independently of one another.

9. System according to claim 8, wherein the control device is designed to successively actuate the individual regions (2.1. 2.2, 2.3) of the magnetic coil assembly in the direction of flow of the body fluid (1) such that a magnetic field is applied in each case by a single region (2.1, 2.2, 2.3) alone to the magnetized body fluid (1).

10. System according to claim 9, wherein the individual regions (2.1, 2.2, 2.3) of the magnetic coil assembly are spatially separated from one another; and
wherein, as seen in the direction of flow of the body fluid (1), the first region (2.1) of the magnetic coil assembly can be actuated by the control device in order to apply a magnetic torque to magnetized or magnetizable particles (1.1) disposed upstream of the first region, and wherein a further region (2.2, 2.3) of the magnetic coil assembly, which is disposed downstream with regard to the first region (2.1) of the magnetic coil assembly, is actuated by the control device if the magnetized or magnetizable particles (1.1) were moved by means of the magnetic field of the first region (2.1) of the magnetic coil assembly along the vessel (3) in the direction of the first region (2.1) of the magnetic coil arrangement.

11. System according to claim 1, wherein the device (2) for applying a magnetic field has a large number of magnetic coil assemblies in successive vessel sections, and wherein a magnetic field applied by the device (2) is generated along the successive vessel sections in such a way that a magnetic force, which has a component pointing in the longitudinal direction of the vessel (3), is exerted on magnetized or magnetizable particles (1.1) in the body fluid (1).

12. System according to claim 1, wherein at least the device (2) for applying a magnetic field can be implanted into the body of a patient.

13. System according to claim 1, wherein the magnetized body fluid includes blood.

14. System according to claim 13, wherein the vessel includes a blood vessel.

15. System according to claim 1, wherein the control device is further designed to temporally vary the strength and/or direction of the magnetic field applied to the magnetized body fluid (1) such that the body fluid (1) moves continuously in the longitudinal direction of the vessel (3).
